(19) United States
(12) Patent Application Publication
(10) Pub. No.: US 2026/0358239 A1
(43) Pub. Date: Nov. 12, 2026

(54) CELL STATE MEASUREMENT METHOD AND CELL STATE MEASUREMENT DEVICE

(71) Applicant: Panasonic Intellectual Property Management Co., Ltd., Osaka (JP)

(72) Inventors: MAYU AIHARA, Osaka (JP); YASUHIRO KABETANI, Osaka (JP); HIDEYUKI TACHIKA, Osaka (JP)

(21) Appl. No.: 19/658,797

(22) Filed: Apr. 27, 2026

(30) Foreign Application Priority Data
May 8, 2025 (JP) 2025-078103

(51) Int. Cl. G01N 21/17; G16B 40/10

(52) U.S. Cl. CPC G01N 21/17; G16B 40/10; G01N 2021/1787

(57) ABSTRACT

The cell state measurement method according to the present disclosure is a method for measuring a cell state of a colony in a container by a temporal variation of a signal intensity of an Optical Coherence Tomography (OCT) signal, including: scanning a measurement light transmitted through an objective lens having an optical axis inclined with respect to a first direction along a plane parallel to a wall surface of the container on the plane, and irradiating a colony, which is located near the wall surface, with the measurement light through the wall surface; detecting a first OCT signal due to optical interference between a signal light that propagates along the optical axis by reflection of the measurement light on the colony and a reference light, at a predetermined time interval; and calculating, based on the detected first OCT signal, a temporal variation characteristic evaluation value of the colony corresponding to irradiation of the plane with a measurement light having an even light intensity.

### BACKGROUND

### 1. Technical Field

The present disclosure relates to a cell state measurement method and a cell state measurement device for cells during culture.

### 2. Description of the Related Art

Optical Coherence Tomography (OCT) is a tomographic imaging method using an interference phenomenon of light, and is used for inspection of a living body or an industrial product, or the like. OCT has recently attracted attention in the field of regenerative medicine because it can acquire optical tomographic images of measurement objects in a non-contact, non-invasive, high-resolution, high-speed, and real-time manner.

In the field of regenerative medicine, along with the development of immunotherapy using cells, industrialization and higher quality of cell culture are required. In the process of culturing cells, a cell cultured body is brought into contact with a wall surface of a culture container and proliferates to form one or more group of cells (referred to as “colony”). In cell culture for several days to several weeks, the quality of a colony changes depending on the culture conditions. In order to ensure the quality of cells, it is required to monitor the process of growth and differentiation of a colony in real time during cell culture and to evaluate the cell state.

As a device for acquiring an optical tomographic image of a cell cultured body during cell culture using OCT and analyzing a cell state includes, for example, the cell state analyzer disclosed in PTL1 is exemplified.

The cell state analyzer disclosed in PTL1 is configured to acquire a tomographic image of a cell cultured body during cell culture using OCT and to analyze the cell state inside the cell cultured body by measuring the shape and volume of the cell cultured body based on the acquired tomographic image.

### CITATION LIST

### Patent Literature

PTL 1: Unexamined Japanese Patent Publication No. 2018-205283

### SUMMARY

A cell state measurement method according to an embodiment of the present disclosure is a method for measuring a cell state of a colony in a container by a temporal variation of a signal intensity of an Optical Coherence Tomography (OCT) signal, including:
scanning a measurement light transmitted through an objective lens having an optical axis inclined with respect to a first direction along a plane parallel to a wall surface of the container on the plane, and irradiating a colony, which is located near the wall surface, with the measurement light through the wall surface;
detecting a first OCT signal due to optical interference between a signal light that propagates along the optical axis by reflection of the measurement light on the colony and a reference light, at a predetermined time interval; and
calculating, based on the detected first OCT signal, a temporal variation characteristic evaluation value of the colony corresponding to irradiation of the plane with a measurement light having an even light intensity.

Furthermore, a cell state measurement device according to an embodiment of the present disclosure is a cell state measurement device of a colony in a container based on a temporal variation of signal intensity of an Optical Coherence Tomography (OCT) signal, including:
a light source device;
an optical system including an objective lens having an optical axis inclined with respect to a first direction along a plane parallel to a wall surface of the container on the plane, which is configured to divide a light emitted from the light source device into a measurement light and a reference light, scan the measurement light on the plane through the objective lens, irradiate a colony, which is located near the wall surface, with the measurement light through the wall surface, and irradiate the reference light on a reference surface;
a detector configured to detect an OCT signal due to optical interference between a signal light that propagates along the optical axis by reflection of the measurement light on the colony and a reference light reflected on the reference surface, at a predetermined time interval; and
an analyzer configured to calculate, based on the OCT signal detected by the detector, a temporal variation characteristic evaluation value of the colony corresponding to irradiation of the plane with a measurement light with an even light intensity.

### BRIEF DESCRIPTION OF THE DRAWINGS

FIG. 1 is a schematic plan view showing a configuration example of a cell state measurement device according to the first exemplary embodiment of the present disclosure;

FIG. 2 is a drawing conceptually illustrating measurement light irradiation of a colony in a culture container in the cell state measurement device of FIG. 1;

FIG. 3A is a drawing conceptually illustrating the relationship between variation of an OCT signal intensity, in which values of OCT signals are expressed in decibels (dB), and a signal light intensity;

FIG. 3B is a drawing that exemplifies temporal variation characteristic values calculated based on OCT signals obtained at a plurality of scan positions associated with one colony;

FIG. 4 is a block diagram illustrating a configuration example of an analyzer for the cell state measurement device according to the first exemplary embodiment of the present disclosure;

FIG. 5 is a flowchart illustrating the cell state measurement process according to the first exemplary embodiment of the present disclosure;

FIG. 6 is a drawing conceptually illustrating a calculation range of the variation characteristic measurement values according to the first exemplary embodiment of the present disclosure;

FIG. 7 is a drawing conceptually illustrating position ranges of colonies in a planar coordinate system;

FIG. 8 is a drawing conceptually illustrating a scan position associated with an individual colony and a corresponding variation characteristic measurement value;

FIG. 9 is a drawing conceptually illustrating the temporal variation characteristic evaluation value of an individual colony calculated by applying correction coefficient data;

FIG. 10 is a flowchart illustrating the correction coefficient data acquisition process according to the first exemplary embodiment of the present disclosure;

FIG. 11 is a diagram conceptually illustrating calculation of the correction coefficient data;

FIG. 12 is a flowchart illustrating a cell state measurement process according to the second exemplary embodiment of the present disclosure; and

FIG. 13 is a diagram conceptually illustrating calculation of a colony thickness in the depth direction according to the second exemplary embodiment of the present disclosure.

### DETAILED DESCRIPTIONS

### Background of Present Disclosure

The device described in PTL1 has a problem that it is difficult to measure the cell state of a colony located near a wall surface of a container.

Therefore, an object of the present disclosure is to provide a cell state measurement method and a cell state measurement device which solve the above-mentioned conventional problems and can measure a cell state of a colony located near a wall surface of a container.

When a cell state during cell culture is to be evaluated, measurement is performed from the outside of a closed culture container so as to maintain sterility. When measurement is performed through the culture container, since an intensity of reflection light from a colony having a small refractive index difference from a culture medium is very small, measurement may be performed by increasing a measurement light intensity. However, the cell state analyzer according to the above-mentioned PTL1 is configured to detect a specular reflection light of a measurement light. In such a configuration, when a measurement light intensity is increased, it is difficult to detect a reflection light signal from a cell cultured body near the wall surface of the culture container due to the influence of a strong specular reflection light from the culture container. Therefore, the present inventors have studied measurement of a cell state of a colony located near a wall surface of a culture container while avoiding the influence of a strong specular reflection light from the container, and have come to the technique according to the following disclosure.

Hereinafter, exemplary embodiments will be described in detail with reference to the drawings as appropriate. However, unnecessary detailed description may be omitted. For example, detailed descriptions of already well-known matters and duplicated descriptions of substantially the same configuration may be omitted. This is to avoid unnecessary redundancy of the following description and to facilitate understanding of those skilled in the art.

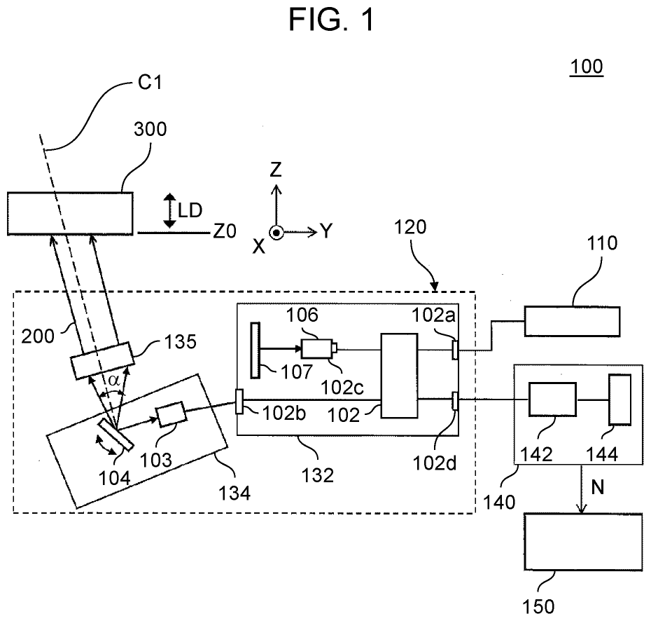

A cell state measurement method and a cell state measurement device according to exemplary embodiments of the present disclosure will be described with reference to FIGS. 1 to 13. The accompanying drawings and the following description are provided for those skilled in the art to fully understand the present disclosure, and are not intended to limit the subject matter described in the claims in any way. Furthermore, in each of the drawings, elements are illustrated exaggeratedly in order to facilitate the description. Note that, in the drawings, substantially the same members are denoted by the same reference numerals.

### First Exemplary Embodiment

### Configuration of Cell State Measurement Device

The configuration of the cell state measurement device according to the first exemplary embodiment of the present disclosure will be described with reference to FIG. 1. FIG. 1 is a schematic plan view illustrating a configuration example of cell state measurement device 100 according to the first exemplary embodiment of the present disclosure.

Cell state measurement device 100 of FIG. 1 is shown in a Y-Z plane and includes light source device 110, optical system 120, detector 140, and analyzer 150, and can acquire an OCT signal of measurement object 300 and analyze the cell state of a colony.

Light source device 110 may be a low coherence light source, and can be configured by, for example, a super luminescent diode (SLD), an ultrashort pulse laser, or a supercontinuum light source, or the like. The light generated from light source device 110 is incident on optical system 120.

Optical system 120 includes optical fiber interferometer 132, measurement head 134, and objective lens 135. Optical fiber interferometer 132 includes coupler 102, collimator lens 106, and reference surface 107, and measurement head 134 includes collimator lens 103 and scanning mechanism 104. Objective lens 135 is disposed between measurement head 134 and measurement object 300.

The light incident on optical fiber interferometer 132 from light source device 110 is guided to coupler 102 by an optical fiber through connection end 102a of coupler 102, and is divided into a measurement light and a reference light in coupler 102. The measurement light is emitted through connection end 102b of coupler 102 and guided to measurement head 134 by an optical fiber. On the other hand, the reference light is guided to collimator lens 106 by an optical fiber through connection end 102c of coupler 102. The reference light emitted from collimator lens 106 is reflected by reference surface 107, enters coupler 102 through collimator lens 106 again, and is guided to detector 140 through connection end 102d of coupler 102.

The measurement light emitted from optical fiber interferometer 132 and guided to measurement head 134 enters scanning mechanism 104 through collimator lens 103. Scanning mechanism 104 may be configured by, for example, a galvano scanner, a polygon scanner, or a resonance scanner, and can scan and emit the measurement light by rotating in two axes. In this exemplary embodiment, for example, scanning mechanism 104 rotates around a rotation axis in the X direction and rotates around a rotation axis in Y direction at a rotation angle α in the Y-Z plane shown in the drawing, so that the measurement light emitted from measurement head 134 can be scanned in X-Y plane. When the measurement range is wide, measurement head 134 itself can be scanned while being driven in at least one of the X direction and Y direction, so that a wide range of X-Y plane can be scanned.

Measurement light 200 emitted from measurement head 134 and emitted through objective lens 135 irradiates measurement object 300 along optical axis C1 of objective lens 135. A part of the measurement light 200 reflected or scattered by measurement object 300 propagates along optical axis C1, enters coupler 102 as a signal light through objective lens 135 and measurement head 134, and is guided to detector 140 through connection end 102d of coupler 102.

Detector 140 includes diffraction grating 142 connected to optical fiber interferometer 132 through connection end 102d, and detector array 144 connected to diffraction grating 142. The measurement light is reflected by the measurement object, and the returned signal light and the reference light returned by reflecting on the reference surface are spectrally divided by diffraction grating 142, the spectrally divided light generated from the signal light and reference light is combined in a plurality of frequency regions to interfere, and a signal of the generated interference light is detected by detector array 144. Thus, the signal of the interference light generated from the signal light and the reference light is measured for each scan position of the measurement light.

In optical fiber interferometer 132, the position of zero point L0 where the measuring path length of the measurement light coincides with the reference path length of the reference light can be adjusted. For example, the position of zero point L0 can be adjusted by changing a distance between collimator lens 106 and reference surface 107 or a propagation distance of an optical fiber between coupler 102 and collimator lens 106. In this exemplary embodiment, a frequency-domain OCT method (also called FD-OCT) capable of high-speed measurement is adopted, and a spectrum of the interference light is Fourier transformed by scanning the measurement light in X-Y plane to obtain the reflection light intensity distribution of each reflecting surface in the depth direction (Z direction) of the measurement object.

Data N of the interference light signal detected by detector 140 is transmitted to analyzer 150, and analyzer 150 calculates a reflectance distribution in each reflective surface of the measurement object and a reflection light intensity distribution in the depth direction of the measurement object based on a signal of the interference light detected at each scan position of the measurement light, thereby a three-dimensional structure of measurement object 300 can be obtained.

In this specification, for convenience of explanation, the signal of the interference light generated by optical interference between the signal light and the reference light at each scan position of the measurement light of the cell state measurement device is referred to as “OCT signal”. In the exemplary embodiment of the present disclosure, although not limited thereto, the plane scanned by the measurement light is X-Y plane, and the depth direction of the measurement object is Z direction.

The configuration of cell state measurement device 100 of the present disclosure is not limited to the configuration example shown in FIG. 1. For example, at least one of optical system 120 and detector 140 may further include other optical elements or may have other configurations.

It is known that dynamic information of a measurement object is measured with ultra-high sensitivity by statistically determining dynamic components of a signal derived from speckle in an OCT signal. In the present disclosure, based on this, a cell state of a colony is measured by obtaining a temporal variation of a signal intensity of an OCT signal within a predetermined time period for the colony during cell culture.

In order to obtain the temporal variation of the signal intensity of the OCT signal for the colony in cell culture, “a temporal variation characteristic value of an OCT signal” is calculated in an exemplary embodiment of the present disclosure. The “temporal variation characteristic value of an OCT signal” is a characteristic value that statistically indicates a temporal variation of a signal intensity of an OCT signal of a colony, and the cell state of the colony can be expressed by the “temporal variation characteristic value of an OCT signal”.

Specifically, in an exemplary embodiment of the present disclosure, an OCT signal is detected at each scan position of a measurement light for a sample containing a colony at a predetermined time interval. For example, in measurement period t, a temporal variation characteristic value of an OCT signal at a scan position (x, y, z) can be calculated as a variance of IdB (x, y, z, ti) within measurement period t, based on a value IdB (x, y, z, ti) represented by a signal intensity in decibels (dB) of the OCT signal detected at the scan position (x, y, z) of the measurement light in a measurement period ti (i=1, 2, . . . N).

As a method for calculating a temporal variation characteristic value of an OCT signal, various numerical calculation methods including, for example, an amount of variation in the OCT signal in a temporal direction and frequency analysis of variation, dispersion value analysis of temporal variation of the signal intensity, and temporal direction differentiation of temporal variation of the signal intensity, and the like can be employed.

Since a signal intensity of an OCT signal of a moving colony (living cells) varies significantly and a signal intensity of an OCT signal of an immobile colony (living cells) varies slightly, the cell state of the colony can be expressed by the temporal variation characteristic value of the OCT signal. In the following description, for convenience of explanation, the “temporal variation characteristic value of an OCT signal” will be simply referred to as “variation characteristic value”.

### Measurement Light Irradiation of Colony in Culture Container

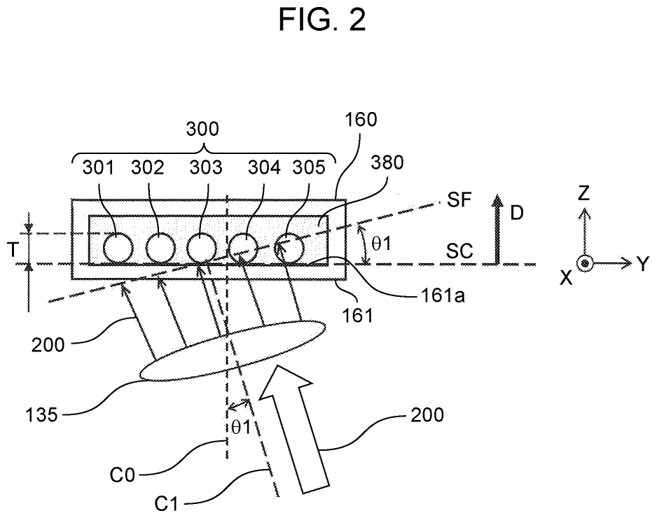

In the present exemplary embodiment, measurement object 300 illustrated in FIG. 1 may be a colony in a container, for example, colony 300 in a culture container. Measurement light irradiation of colony 300 in a culture container will be described with reference to FIG. 2. FIG. 2 is a diagram conceptually illustrating measurement light irradiation of colony 300 in a culture container in cell state measurement device 100 shown in FIG. 1.

As shown in FIG. 2, colony 300 exists in culture medium 380 in culture container 160, and individual colonies 301 to 305 contained in colony 300 are located near wall surface 161 of culture container 160 and are arranged so as to contact inner side 161a of wall surface 161. In the case of planar culture, wall surface 161 of culture container 160 may be flat, and individual colonies 301 to 305 grow along wall surface 161 and have colony distribution surface SC (X-Y plane) parallel to wall surface 161 and a colony thickness along depth direction D (Z direction).

In the present exemplary embodiment, as illustrated in the drawing, optical axis C1 of objective lens 135 is inclined with respect to Y direction illustrated in the drawing and disposed at angle θ1 with respect to direction C0 orthogonal to wall surface 161. Measurement light 200 transmitted through objective lens 135 propagates along optical axis C1 and enters wall surface 161 with inclination. Since measurement light 200 enters wall surface 161 inclined with respect to Y direction, it is possible to suppress a strong specular reflection light from wall surface 161 from entering a signal light propagating along optical axis C1. As a result, a ratio of the reflected or scattered light by colony 300 near wall surface 161 in the signal light increases, and thus the measurement accuracy of the colony near the wall surface can be improved.

In the present specification, “inclined with respect to Y direction” or “with inclination with respect to the wall surface” means a direction that is not orthogonal to Y direction or the wall surface.

Angle θ1 can be configured by an angle equal to or greater than an opening angle of objective lens 135 and less than 90 degrees. The opening angle of the objective lens indicates half of a condensing angle of the objective lens, that is, the maximum angle of a light that enters the objective lens from an image side with respect to an optical axis of the objective lens. For example, when an opening angle of objective lens 135 is θ0, numerical aperture NA of objective lens 135 is NA=n×sinθ0, and angle θ1 of optical axis C1 with respect to direction C0 satisfies θ0≤θ1<90°. Thus, the influence of the specular reflection light from wall surface 161 can be suppressed, and the measurement accuracy of colonies near the wall surface can be improved.

Measurement light 200 that enters wall surface 161 (X-Y plane in this exemplary embodiment) with inclination scans along the colony distribution plane SC in X-Y plane, and converges on focal plane SF forming angle θ1 with respect to the colony distribution plane SC. Therefore, the light intensity of measurement light 200 varies in Y direction of X-Y plane with the scanning of measurement light 200, and unevenness of the generated signal light intensity occurs.

Although FIG. 2 shows that optical axis C1 of objective lens 135 is disposed as inclined with respect to Y direction, the present disclosure is not limited to this. Optical axis C1 of objective lens 135 may be disposed with an inclination with respect to either or both of X and Y directions, for example.

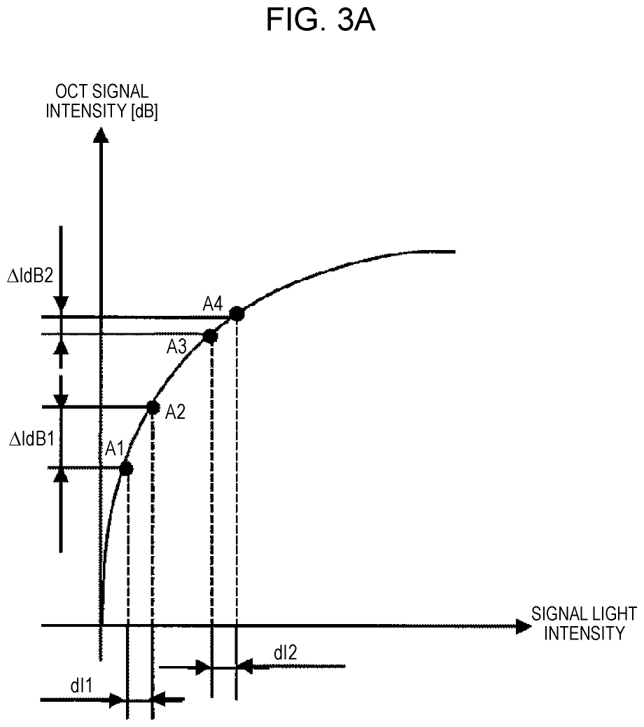

The variation of a signal intensity of an OCT signal due to unevenness of a signal light intensity generated in X-Y scan plane of measurement light 200 will be described with reference to FIGS. 3A and 3B. FIG. 3A is a diagram conceptually illustrating the relationship between variation of an OCT signal intensity, in which values of OCT signals are expressed in decibels (dB), and a signal light intensity, and FIG. 3B is a diagram illustrating temporal variation characteristic values calculated based on OCT signals obtained at a plurality of scan positions related to one colony.

As shown in FIG. 3A, for example, in measurement values A1 to A4 of OCT signals having different signal light intensities, signal light intensity difference dI1 between measurement value A1 and measurement value A2 measured at a lower signal light intensity is substantially equal to signal light intensity difference dI2 between measurement value A3 and measurement value A4 measured at a higher signal light intensity, and the signal light intensity difference dI1 and the signal light intensity difference dI2 are in relationship dI1=dI2. At this time, variation ΔIdB2 of the OCT signal intensity between A3 and A4 measured at a higher signal light intensity is smaller than variation ΔIdB1 of the OCT signal intensity between A1 and A2 measured at a lower signal light intensity.

As described above, the variation characteristic values calculated based on the OCT signals measured at different scan positions vary during the cell state measurement of a colony due to the unevenness of the signal light intensity caused by the measurement light that enters X-Y plane with inclination. As shown in FIG. 3B, if X-Y scan plane is irradiated with an even measurement light, the variation characteristic values indicating the cell state of one colony are constant. On the other hand, when a light intensity of a measurement light varies in Y direction of X-Y scan plane, respective variation characteristic values MV1 to MV5 calculated based on OCT signals obtained at scan positions P1 to P5 become different values.

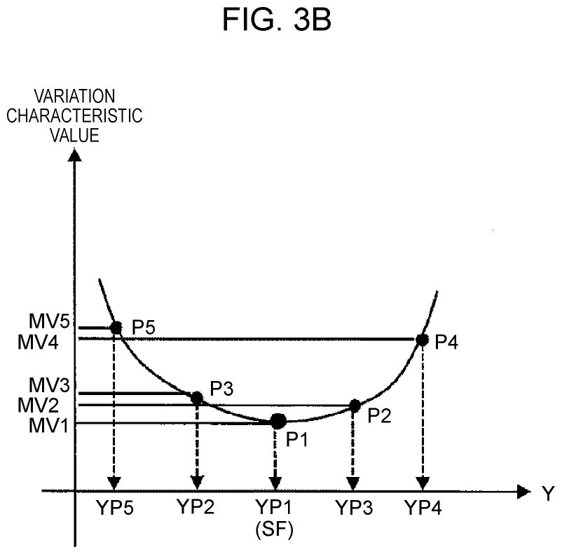

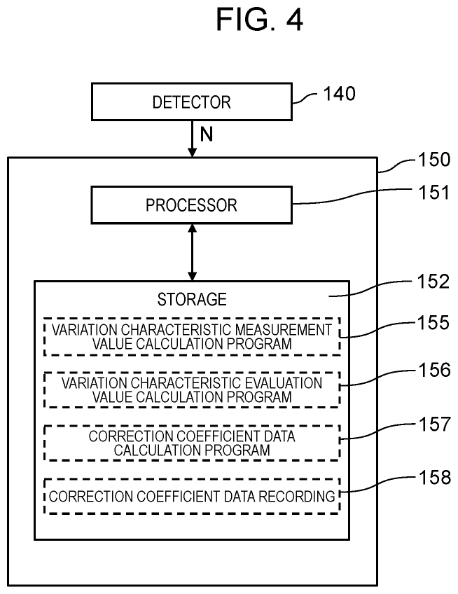

Furthermore, for example, when a position range of a colony illustrated in FIG. 3B includes scan position P1 near focal plane SF of a measurement light, variation characteristic value MV1 calculated based on the OCT signal obtained at scan position P1 with the strongest signal light is the smallest, and variation characteristic values MV2 to MV5 calculated based on the obtained OCT signals gradually increase as the distance of the scan position away from focal plane SF in Y direction increases. As described above, due to unevenness of the signal light intensity due to the variation of the measurement light intensity in X-Y plane, the calculated variation characteristic value cannot accurately indicate the cell state of the colony. Therefore, in the present disclosure, analyzer 150 calculates a variation characteristic value of an OCT signal by irradiation of X-Y plane with a measurement light having an even light intensity from an OCT signal obtained under irradiation of a measurement light entering X-Y plane with inclination. Thus, the cell state of the colony can be accurately measured. Hereinafter, operations of analyzer 150 will be described with reference to FIGS. 4 and 5.

In the following description, for convenience of explanation, a variation characteristic value calculated based on an OCT signal detected by detector 140 will be referred to as “variation characteristic measurement value (MV)”, and a variation characteristic value of an OCT signal by irradiation of X-Y plane with a measurement light with an even light intensity will be referred to as “variation characteristic evaluation value (EV)”. Furthermore, data of a correction coefficient (cf) used for calculating “variation characteristic evaluation value (EV)” based on “variation characteristic measurement value (MV)” will be referred to as “correction coefficient data”.

### Analyzer

Analyzer 150 of cell state measurement device 100 of the present disclosure can calculate a variation characteristic measurement value (MV) based on an OCT signal detected by detector 140, obtain correction coefficient data, and apply a correction coefficient (cf) to the calculated variation characteristic measurement value (MV) to calculate a variation characteristic evaluation value (EV).

FIG. 4 is a block diagram showing a configuration example of analyzer 150 of cell state measurement device 100 according to the first exemplary embodiment of the present disclosure. Analyzer 150 includes processor 151 and storage 152. Analyzer 150 realizes predetermined functions by processor 151 executing commands stored in storage 152. The function of analyzer 150 may be configured only by hardware or may be realized by combining hardware and software. Analyzer 150 may include one or more processors 151.

Processor 151 may be configured by, for example, a microcomputer, a CPU (Central Processing Unit), an MPU (Micro Processing Unit), a GPU (Graphics Processing Unit), a DSU (Digital Signal Unit), an FPGA (Field Programmable Gate Array), an ASIC (Application Specific Integrated Circuit), and the like. Processor 151 may also be configured by a dedicated electronic circuit designed to realize predetermined functions.

Storage 152 is a storage medium for storing programs and data for realizing the functions of analyzer 150. Storage 152 can be realized by, for example, an HDD (Hard Disk Drive), an SSD (Solid State Drive), an RAM (Random Access Memory), a DRAM (Dynamic Random Access Memory), a ferroelectric memory, a flash memory, a magnetic disk, or a combination of these HDD, SSD, RAM, DRAM, ferroelectric memory, flash memory and magnetic disk.

For example, data N of an OCT signal received from detector 140 can be stored in storage 152. Furthermore, various data calculated based on data N of the OCT signal can be stored in storage 152. Note that the transmission and reception of signals by analyzer 150 may be realized by wired connection, or may be realized by wireless transfer.

Storage 152 stores programs and data including variation characteristic measurement value calculation program 155, variation characteristic evaluation value calculation program 156, correction coefficient data calculation program 157, and correction coefficient data record 158. Note that when analyzer 150 is connected to a network, these programs and data may be downloaded from the network as required.

Variation characteristic measurement value calculation program 155, variation characteristic evaluation value calculation program 156, and correction coefficient data calculation program 157 can be executed by processor 151 in a cell state measurement process and a correction coefficient data acquisition process according to an exemplary embodiment of the present disclosure. Correction coefficient data record 158 is formed by executing correction coefficient data calculation program 157, and is utilized by variation characteristic evaluation value calculation program 156. Cell state measurement process 400, correction coefficient data acquisition process 500, and the operations of analyzer 150 will be described below with reference to FIGS. 5 to 11.

### Cell State Measurement Process

FIG. 5 is a flowchart illustrating cell state measurement process 400 according to the first exemplary embodiment of the present disclosure.

In step S401 shown in FIG. 5, as illustrated in FIGS. 1 and 2, measurement light 200 transmitted through objective lens 135 having optical axis C1 inclined with respect to Y direction scans in X-Y plane and irradiates the colonies in culture container 160 along optical axis C1 through wall surface 161.

The colonies in culture container 160 include, for example, colonies in planar culture, and individual colonies may grow along inner wall surface 161a (FIG. 2) of culture container 160 and the individual colonies may be in a state where they are spreading over the entirety of colony distribution surface SC to such an extent that the colonies do not come into contact with each other. Although not limited to this, in the present exemplary embodiment, iPS cells may be used as the colonies, for example.

Next, in step S402, OCT signals of the colony due to optical interference between a signal light and a reference light are detected at predetermined time intervals for the respective scan positions of measurement light 200 on X-Y plane by detector 140. The data of the detected OCT signals is transmitted to analyzer 150.

Subsequently, steps S403 to S407 are executed by analyzer 150. In steps S403 to S405, processor 151 executes variation characteristic measurement value calculation program 155. In step S406, processor 151 may execute correction coefficient data calculation program 157 to acquire the correction coefficient data, or correction coefficient data record 158 may be read from storage 152 to acquire correction coefficient data. In step S407, processor 151 executes variation characteristic evaluation value calculation program 156.

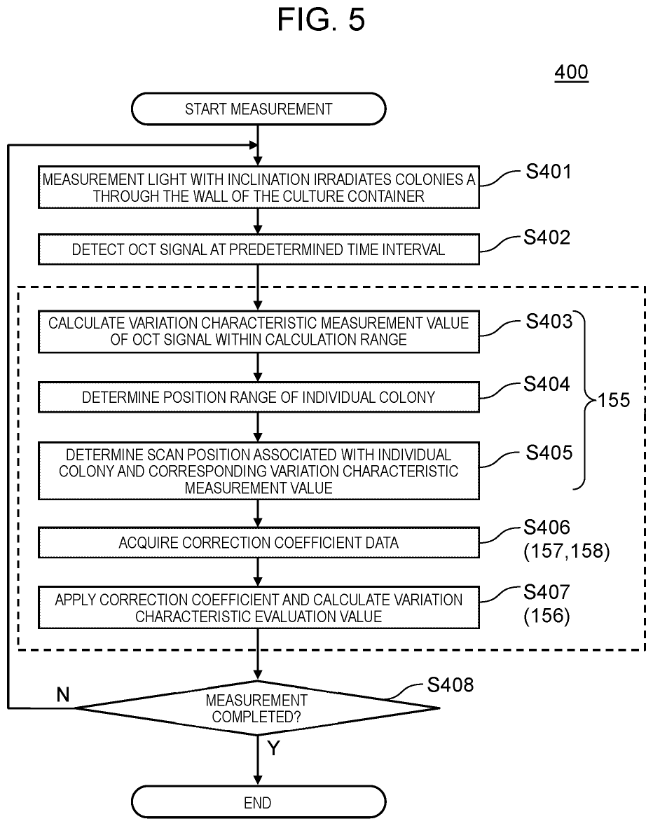

In step S403, a variation characteristic measurement value (MV) within a predetermined range is calculated based on the data of the OCT signal received from detector 140. At this time, in Y-Z plane, an OCT signal within a calculation range for variation characteristic measurement values is extracted in depth direction D illustrated in FIG. 2 from inner wall surface 161a of culture container 160 to calculate the variation characteristic measurement value (MV). This will be described with reference to FIG. 6.

FIG. 6 is a diagram conceptually illustrating the calculation range for variation characteristic measurement values according to the first exemplary embodiment of the present disclosure. As illustrated, planar-grown colony 300 including colonies 300a, 300b and 300c growing along inner wall surface 161a, and floating cells or floating colony 360 including colonies 360a, 360b and 360c apart from inner wall surface 161a are present in culture container 160. Planar-grown colonies 300 are normal cells in culture (living cells), and floating cells or floating colony 360 are dead cells. Since OCT signals generated by floating cells or floating colony 360 cause noise in the measurement of a cell state, in the present exemplary embodiment, a variation characteristic measurement value (MV) is calculated by removing the OCT signals generated by floating cells or floating colony 360.

For example, when iPS cells are used, colonies have a thickness of about 10 μm, and colonies in the planar culture have a monolayer cell structure. Therefore, for example, a range of about 20 μm along depth direction D from inner wall surface 161a of culture container 160 is set as calculation range H for a variation characteristic measurement value (MV), and the variation characteristic measurement value (MV) can be calculated by extracting an OCT signal within calculation range H.

In the spectrometer-type OCT method (SD-OCT), which is a kind of frequency-domain OCT method, OCT signals in a range of several millimeters are usually detected in depth direction D. By extracting OCT signals in range H of about 20 μm, measurement noise due to floating cells or floating colonies can be removed and the burden of data processing can be reduced.

The present disclosure is not limited to the type of the colony as a measurement object. Calculation range H of the variation characteristic measurement value (MV) can be set depending on the type of cells as a measurement object.

In step S403, for example, the position of inner wall surface 161a of culture container 160 in depth direction D (Z direction) can be obtained by comparing signal light intensities within a predetermined range, and specifying a generation position of a local peak signal intensity with regard to an intensity distribution of a signal light in Z direction. Furthermore, an approximate thickness of the colony can be obtained based on a range having a signal intensity equal to or greater than a predetermined threshold value, from the position of inner wall surface 161a toward depth direction D. Moreover, for example, a range of about 2 times the thickness of the colony from the position of inner wall surface 161a can be set as calculation range H of the variation characteristic measurement value (MV).

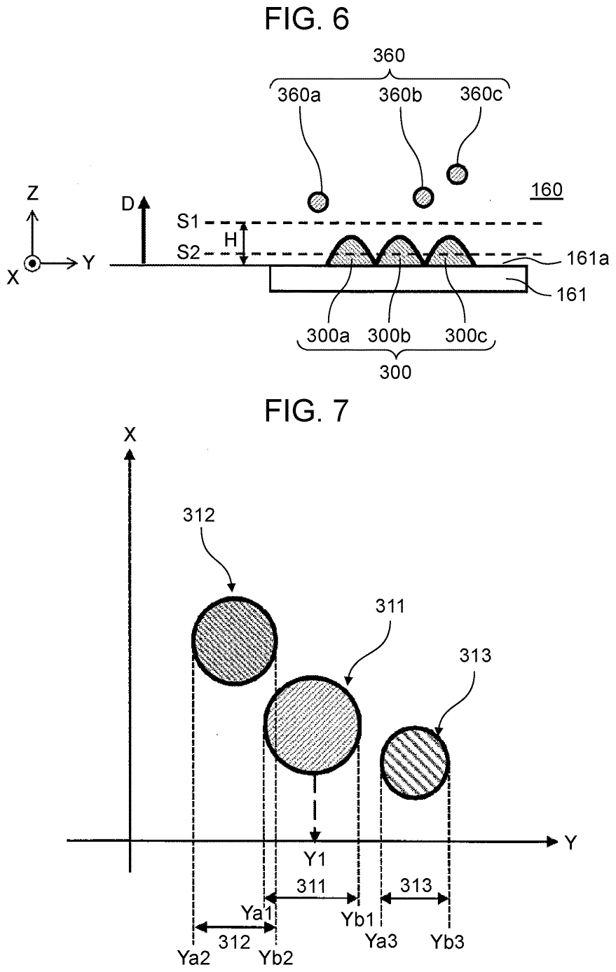

Next, in step S404, the position range of the individual colony is determined. For example, the position range of the individual colony can be determined based on an OCT signal within calculation range H. For example, a plane image of a colony in plane S2 in range H in depth direction D shown in FIG. 6 is formed, and the position range of said colony can be determined based on the external shape of the individual colony. An example will be described with reference to FIG. 7. FIG. 7 is a diagram conceptually illustrating the position ranges of colonies in a planar coordinate system.

As shown in FIG. 7, in planar coordinate system X-Y, the position ranges of colonies 311, 312, 313 in Y direction are determined by Ya1-Yb1, Ya2-Yb2, Ya3-Yb3, respectively. The position ranges of colonies 311, 312, 313 in X direction can be determined similarly. Furthermore, the position range of the individual colony in the depth direction can be determined based on an OCT signal.

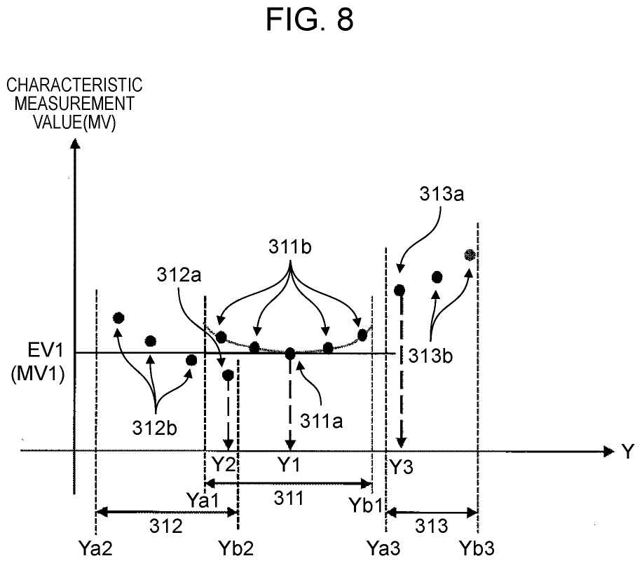

Subsequently, in step S405, the scan position of the measurement light associated with the individual colony, and the corresponding variation characteristic measurement value (MV) calculated in corresponding step S403 are determined based on the position range of the individual colony determined in step S404. This will be described with reference to FIG. 8. FIG. 8 is a diagram conceptually illustrating scan positions, each of which is associated with the individual colony, and corresponding variation characteristic measurement values.

As shown in FIG. 8, in planar coordinate system X-Y, the variation characteristic measurement values (MV) calculated for scan positions 311a, 311b fall within position range Ya1-Yb1 of the colony 311 in Y direction, and are associated with colony 311. The variation characteristic measurement values (MV) calculated for scan positions 312a, 312b fall within range Ya2-Yb2 of colony 312 in Y direction and are associated with colony 312. The variation characteristic measurement values (MV) calculated for scan positions 313a, 313b fall within range Ya3-Yb3 of colony 313 in Y direction and are associated with colony 313. In this way, in planar coordinate system X-Y, the scan position associated with the individual colony is determined, and the variation characteristic measurement value (MV) corresponding to each of the scan positions can be obtained.

Subsequently, in step S406, correction coefficient data is acquired. At this time, when correction coefficient data record 158 is stored in storage 152, correction coefficient data can be acquired by reading correction coefficient data record 158 from storage 152. When correction coefficient data record 158 is stored in storage 152, correction coefficient data can be formed by making processor 151 execute correction coefficient data calculation program 157, and calculating a correction coefficient.

The correction coefficient (cf) for calculating the variation characteristic evaluation value (EV) from the variation characteristic measurement value (MV) is used to correct the deviation of the variation characteristic value caused by the measurement light that enters with inclination, and does not depend on the measurement object. Therefore, the correction coefficient data can be acquired in advance by using an object different from the object used in the cell state measurement before executing cell state measurement process 400 in FIG. 5, and stored in storage 152, so that the correction coefficient data can be utilized in step S406. Furthermore, the correction coefficient data can also be formed by executing correction coefficient data calculation program 157 in step S406 during execution of cell state measurement process 400. The formed correction coefficient data is stored in storage 152 as correction coefficient data record 158, and can be used in further cell state measurement for the same sample or in cell state measurement for another sample. Formation of the correction coefficient data will be described later in detail.

Subsequently, in step S407, the correction coefficient (cf) based on the correction coefficient data acquired in step S406 is applied to the variation characteristic measurement value (MV) associated with the individual colony determined in step S405 to calculate the variation characteristic evaluation value (EV) of the individual colony.

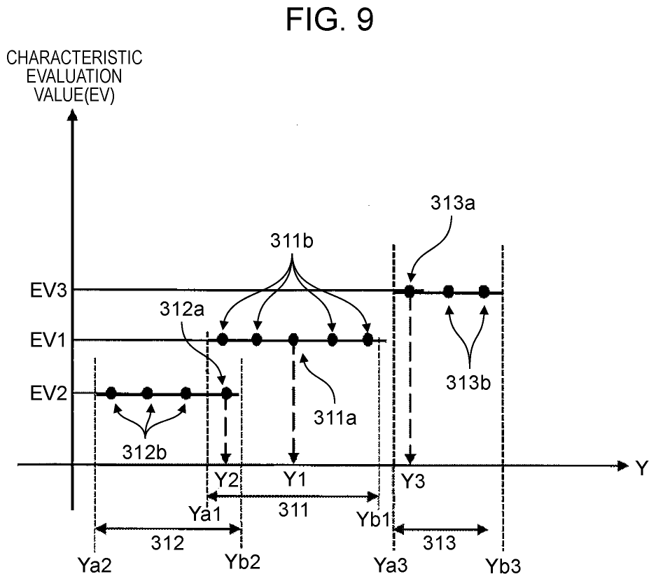

FIG. 9 is a drawing conceptually illustrating the temporal variation characteristic evaluation value of the individual colony calculated by applying the correction coefficient data. As shown in FIG. 9, temporal variation characteristic evaluation value EV1 of colony 311 is calculated by applying a corresponding correction coefficient (CV) to variation characteristic measurement values (MV) corresponding to scan position 311a and 311b associated with colony 311. Similarly, for colonies 312, 313, respective variation characteristic evaluation values EV2 and EV3 are calculated by applying corresponding correction coefficients (CV).

In step S408, the above-mentioned steps S401 to S407 can be repeatedly executed. By measuring the cell state in this way, the process of growth and differentiation of colonies can be monitored and evaluated in real time during cell culture.

### Correction Coefficient Data Acquisition Process

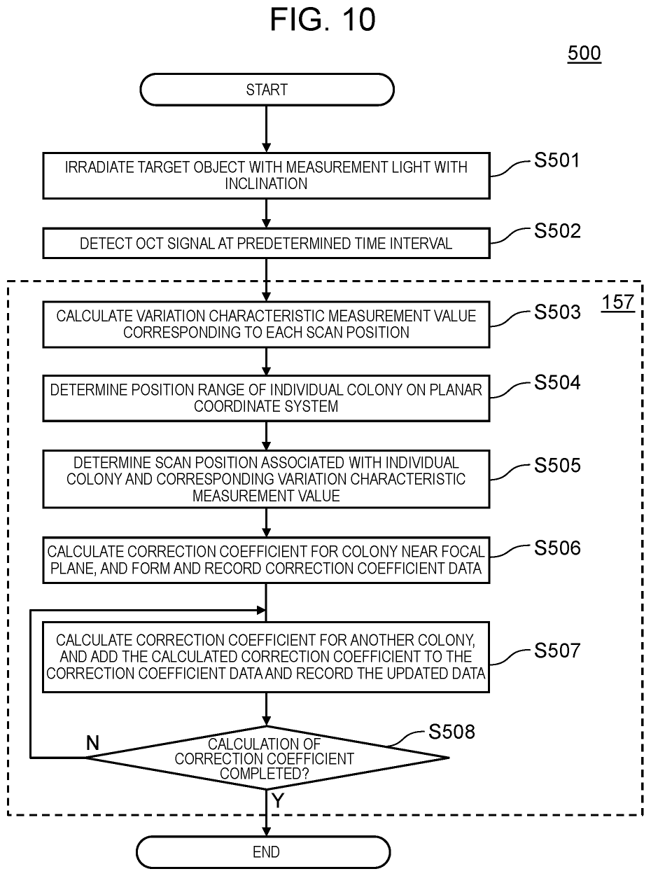

A correction coefficient data acquisition process will be described with reference to FIG. 10. FIG. 10 is a flowchart showing correction coefficient data acquisition process 500 according to the first exemplary embodiment of the present disclosure. Step S503 to step S508 of correction coefficient data acquisition process 500 can be performed by causing processor 151 to execute correction coefficient data calculation program 157 in analyzer 150.

Correction coefficient data acquisition process 500 may be executed, for example, by cell state measurement device 100 used in a cell state measurement process, and may be executed for the same measurement object as the measurement object of cell state measurement process 400, or may be executed for a measurement object different from the measurement object of cell state measurement process 400. The measurement object of correction coefficient data acquisition process 500 may be, for example, a biological sample including cells such as a colony, or a stationary object not including cells such as a colony, for example, a container. This is because, even if the measurement object is different, the measurement light that enters with inclination of cell state measurement device 100 causes a similar variation in variation characteristic value.

In the following description of correction coefficient data acquisition process 500, for convenience of description, colonies will be described as measurement objects with reference to FIGS. 7 to 9 illustrating colonies. However, it should be understood that the present disclosure is not limited thereto. Therefore, correction coefficient data acquisition process 500 may be executed on an object including a plurality of partial zones arranged along X-Y plane instead of a biological sample including colonies in the following description as the measurement object. In this specification, “partial zone” may be living cells such as colonies in a biological sample as a measurement object, or a part occupying a partial region in a stationary object as a measurement object.

As shown in FIG. 10, steps S501 and S502 of correction coefficient data acquisition process 500 may be performed similarly to steps S401 and S402 of cell state measurement process 400 of FIG. 5, and a detailed description thereof is omitted.

In step S503, a variation characteristic measurement value (MV) is calculated based on the OCT signal detected for each scan position of the measurement light. Since the variation of light intensity in X-Y plane due to the measurement light that enters with inclination is generally constant within the thickness range of the colony, the correction coefficient data calculated in one X-Y plane can be used to calculate a variation characteristic value (EV) within the thickness range of the colony in the depth direction (Z direction).

In step S504, for example, as shown in FIG. 7, the position range of each individual colony is determined in the planar coordinate system X-Y.

In step S505, as shown in FIG. 8, scan positions associated with respective colonies 311, 312, 313 and the corresponding variation characteristic values (EV) are determined in planar coordinate system X-Y.

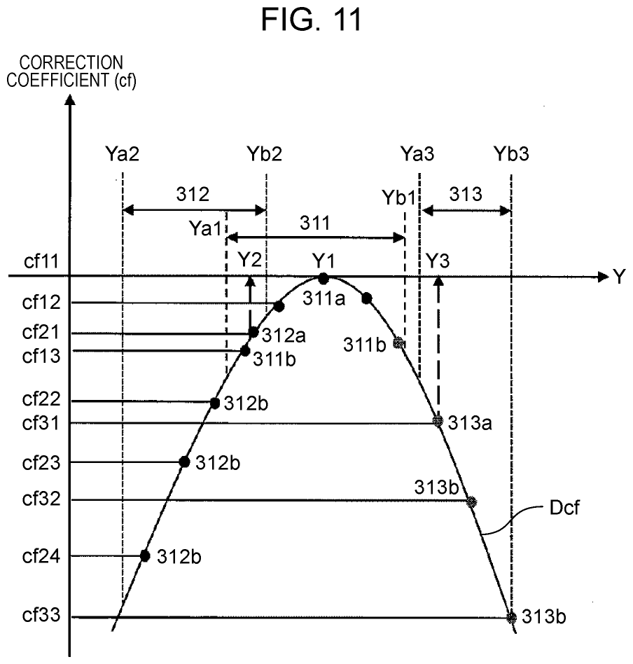

Subsequently, in step S506, correction coefficients are calculated for the colonies near the focal plane, and correction coefficient data is formed and recorded. Calculation of the correction coefficients and formation of the correction coefficient data will be described with reference to FIG. 11 in addition to FIGS. 8 and 9. FIG. 11 is a diagram conceptually illustrating calculation of the correction coefficient data.

At this time, the scan position near focal plane SF of the measurement light can be determined in planar coordinate system X-Y on the basis of the light intensity of the signal light returned by propagating along optical axis C1. As shown in FIG. 8, for example, when the light intensity of the signal light is the strongest at scan position 311a related to colony 311, it is understood that colony 311 is located near focal plane SF. Variation characteristic measurement value MV1 corresponding to scan position 311a can be set as variation characteristic evaluation value EV1 of colony 311 (FIG. 9), and correction coefficient (cf) corresponding to position coordinate Y1 in Y direction of scan position 311a can be calculated. In this exemplary embodiment, correction coefficient (cf) is calculated as a ratio between the corresponding variation characteristic evaluation value (EV) and the variation characteristic measurement value (MV), and correction coefficient cf11 corresponding to position coordinate Y1 (FIG. 11) is calculated as 1.

Next, correction coefficients cf12, cf13 (FIG. 11) and the like corresponding to the position coordinates in Y direction of each scan position 311b related to colony 311 can be calculated so that all of the variation characteristic measurement values MV corresponding to the other scan positions 311b related to colony 311 are equal to variation characteristic evaluation value EV1. By calculating the correction coefficients (cf) for colony 311, correction coefficient data Dcf is formed in the range of position coordinates Ya1 to Yb1 in Y direction shown in FIG. 11.

Correction coefficient data Dcf can be formed based on calculated correction coefficients cf11, cf12, cf13, and the like. Correction coefficient data Dcf is data indicating the correlation between a position coordinate in Y direction and a correction coefficient (cf) in planar coordinate system X-Y of the scan plane. Correction coefficient data Dcf as formed is recorded and stored in storage 152 as correction coefficient data record 158.

Note that the present disclosure is not limited to the calculation method in which the variation characteristic measurement value MV1 corresponding to scan position 311a having the strongest light intensity of the signal light is used as variation characteristic evaluation value EV1. For example, an average value of variation characteristic measurement values corresponding to a plurality of scan positions having a predetermined light intensity of the signal light may be used as the variation characteristic evaluation value.

Next, a correction coefficient is calculated for a colony other than the colonies near the focal plane. For example, calculation of a correction coefficient will be described for colony 312 partially overlapping with colony 311 shown in FIG. 8. Correction coefficient cf21 corresponding to position coordinate Y2 of scan position 312a in the overlapping region between colony 312 and colony 311 in planar coordinate system X-Y is included in recorded correction coefficient data Dcf formed in step S506. At this time, correction coefficient cf21 obtained based on correction coefficient data Dcf in FIG. 11 is applied to the variation characteristic measurement value corresponding to scan position 312a to calculate variation characteristic evaluation value EV2 of colony 312 (FIG. 9). Next, correction coefficients cf21, cf22, cf23, cf24, etc. and the like corresponding to the position coordinate in Y direction of each scan position 312b related to colony 312 can be calculated so that all of the variation characteristic measurement values MV corresponding to the other scan positions 312b related to colony 312 are equal to variation characteristic evaluation value EV2 (FIG. 11). Calculated correction coefficients cf21, cf22, cf23, cf24, and the like are added to correction coefficient data Dcf formed in step S506 and recorded. By calculating correction coefficients (cf) for colonies 311, 312 in this way, correction coefficient data Dcf is formed in the range of position coordinates Ya2 to Yb1 in Y direction shown in FIG. 11.

Next, calculation of a correction coefficient will be described for colony 313 that is not overlapping with colonies 311, 312 shown in FIG. 8. In planar coordinate system X-Y, correction coefficient cf31 corresponding to position coordinate Y3 in Y direction of scan position 313a related to colony 313 can be obtained based on an extension line of correction coefficient data Dcf formed in step S506 in the range from position coordinate Ya1 to position coordinate Yb1. The obtained correction coefficient cf31 is applied to a variation characteristic measurement value corresponding to scan position 313a to calculate variation characteristic evaluation value EV3 of colony 313 (FIG. 9). Furthermore, correction coefficients cf31, cf32, cf33, and the like corresponding to the position coordinate in Y direction of each scan position 313b related to colony 313 are calculated so that all of the variation characteristic measurement values MV corresponding to the other scan positions 313b related to colony 313 are equal to variation characteristic evaluation value EV3 (FIG. 11). The calculated correction coefficients cf31, cf32, cf33 and the like are further added to correction coefficient data Dcf and recorded. By calculating correction coefficients (cf) for colonies 311, 312, 313 in this way, correction coefficient data Dcf is formed in the range of position coordinates Ya2 to Yb3 in Y direction shown in FIG. 11.

Correction coefficient data Dcf corresponding to all the position coordinates in Y direction of X-Y plane can be formed and recorded by repeatedly executing from steps S503 to S508.

Note that the present disclosure is not limited to the method for calculating correction coefficient (cf) or the format of correction coefficient data Dcf. Correction coefficient (cf) is not limited to calculation as a ratio between the corresponding variation characteristic evaluation value (EV) and the variation characteristic measurement value (MV), but can also be calculated by other arithmetic expressions. Correction coefficient data Dcf may be, for example, a table showing correlation between position coordinates in Y direction and correction coefficient (cf), or may be a quadratic function curve or an n-order function curve.

Furthermore, in the above-mentioned description, the correction coefficient (cf) and correction coefficient data Dcf corresponding to the position coordinate in Y direction have been described. However, when optical axis C1 of objective lens 135 is disposed as inclined with respect to X direction, a correction coefficient corresponding to the position coordinate in X direction can be similarly calculated, and correction coefficient data can be similarly formed.

As described above, in the cell state measurement device according to the first exemplary embodiment of the present disclosure, the influence of the specular reflection light from the wall surface can be suppressed by entering the measurement light at an inclination with respect to the wall surface of the container, and the measurement accuracy of the colony near the wall surface can be improved. Furthermore, the variation characteristic evaluation value of a colony by irradiating a measurement light with an even light intensity in X-Y plane can be calculated, and the cell state of the colony can be indicated. Therefore, the cell state of the colony located near the wall surface of the container can be measured.

### Second Exemplary Embodiment

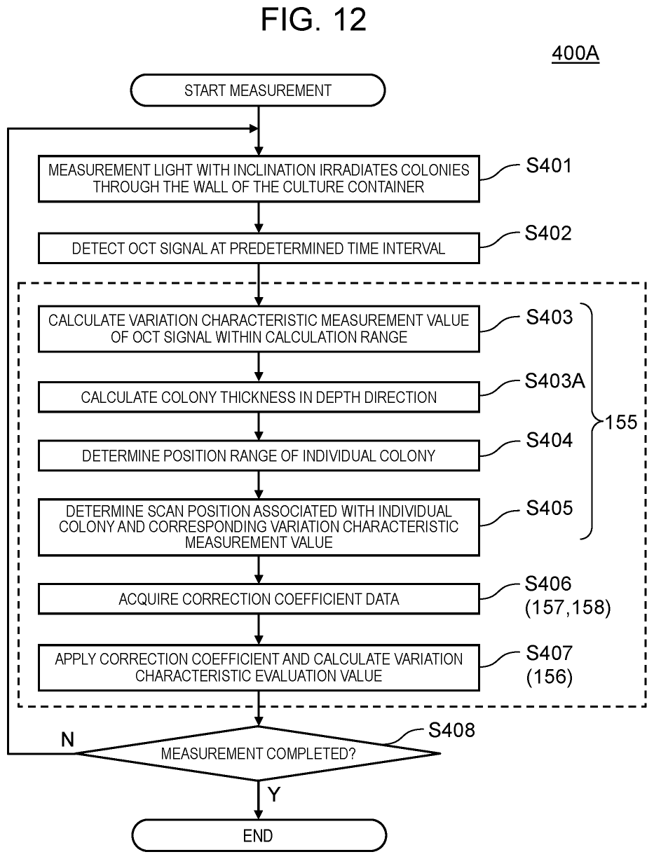

A cell state measurement process according to the second exemplary embodiment of the present disclosure will be described with reference to FIG. 12. FIG. 12 is a flowchart showing cell state measurement process 400A according to the second exemplary embodiment of the present disclosure.

Cell state measurement process 400A shown in FIG. 12 is different from cell state measurement process 400 shown in FIG. 5 in that it further includes step 403A, and the other steps are processed in the same manner as in cell state measurement process 400. Hereinafter, the same parts as in cell state measurement process 400 are omitted, and the description will focus on step 403A.

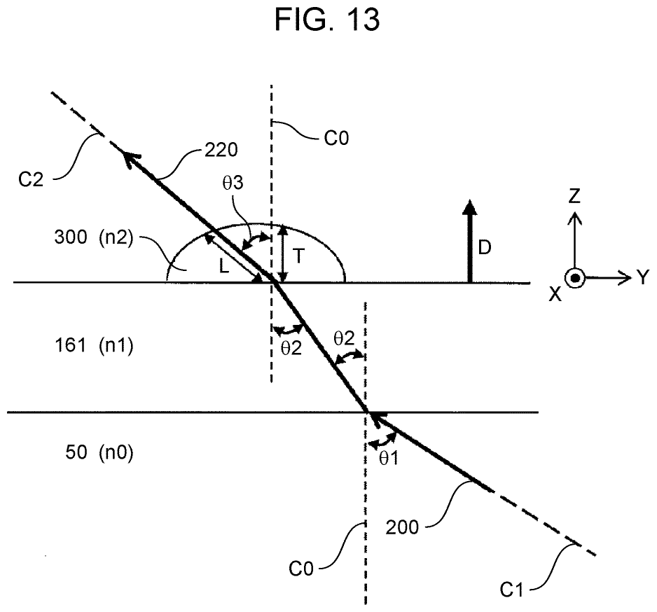

In step 403A, a colony thickness is calculated. Calculation of the colony thickness in step 403A is included in variation characteristic measurement value calculation program 155 and may be conducted by making processor 151 execute the calculation. The calculation of the colony thickness will be described with reference to FIG. 13. FIG. 13 is a diagram conceptually illustrating the calculation of the colony thickness in the depth direction (Z direction) according to the second exemplary embodiment of the present disclosure.

When cell state measurement is performed using cell state measurement device 100 shown in FIG. 1, measurement light 200 enters wall surface 161 of a container with inclination along optical axis C1, and propagates through optical path length L in colony 300, as shown in FIG. 13. At this time, the thickness of the colony cross-sectional structure calculated based on an OCT signal is optical path length L in the colony, which is different from colony thickness T in depth direction D (Z direction).

In step 403A, colony thickness T in depth direction D is calculated by correcting the variation of the optical path length caused by the irradiation of measurement light 200 incident with inclination.

Measurement light 200 passes through the air, passes through wall surface 161 of the culture container, and then enters colony 300. Here, the refractive index of the air is n0, the refractive index of the culture container is n1, and the refractive index of the colony is n2. When measurement light 200 enters at angle θ1 with respect to direction C0 orthogonal to wall surface 161 and at angle θ3 with respect to direction C0, colony thickness T is calculated by the following equations (1) and (2) according to the Snell's law.

[Equation 1]

sin θ₂=n₀/n₂×sin θ₀ (1)

[Equation 2]

T=L×sin(90−θ₂) (2)

By calculating colony thickness T in depth direction D, the position range of the individual colony can be more accurately determined in a three-dimensional manner in step S404. Based on this, for example, the temporal variation characteristic evaluation values (EV) of colonies can be calculated in a plurality of X-Y planes within colony thickness T range in depth direction D (Z direction), and the cell state of the individual colony can be measured in a three-dimensional manner.

As described above, in the cell state measurement device according to the second exemplary embodiment of the present disclosure, the influence of a specular reflection light from the wall surface can be suppressed by entering the measurement light inclined with respect to the wall surface of the container, and the measurement accuracy of a colony near the wall surface can be improved. Furthermore, a variation characteristic evaluation value of a colony by irradiating a measurement light irradiation with an even light intensity on X-Y plane and a colony thickness in the depth direction (Z direction) can be calculated to indicate a cell state of the colony in a three-dimensional manner. Therefore, the cell state of the colony located near the wall surface of the container can be measured.

### Other Exemplary Embodiments

### (Supplementary Note)

The above description of the exemplary embodiments discloses the following techniques.

(Technique 1) A cell state measurement method for measuring a cell state of a colony in a container by a temporal variation of a signal intensity of an Optical Coherence Tomography (OCT) signal, the cell state measurement method including:
scanning a measurement light transmitted through an objective lens having an optical axis inclined with respect to a first direction along a plane parallel to a wall surface of the container on the plane, and irradiating a colony, which is located near the wall surface, with the measurement light through the wall surface;
detecting a first OCT signal due to optical interference between a signal light that propagates along the optical axis by reflection of the measurement light on the colony and a reference light, at a predetermined time interval; and
calculating, based on the detected first OCT signal, a temporal variation characteristic evaluation value of the colony corresponding to irradiation of the plane with a measurement light having an even light intensity.

By this method, the cell state of the colony located near the wall surface of the container can be measured.

(Technique 2) The cell state measurement method according to Technique 1,
wherein the optical axis forms a first angle with respect to a direction orthogonal to the wall surface, and
the first angle is an angle equal to or greater than an opening angle of the objective lens and less than 90 degrees.

By this method, the influence of a specular reflection light from the wall surface can be suppressed, and the measurement accuracy of the colony near the wall surface can be improved.

(Technique 3) The cell state measurement method according to Technique 1 or 2,
wherein the calculating the temporal variation characteristic evaluation value comprises:
determining a calculation range from the wall surface toward the inside of the container in a direction orthogonal to the wall surface; and
calculating the temporal variation characteristic evaluation value of the colony based on the first OCT signal within the calculation range determined in determining.

By this method, the cell state of a colony located near the wall surface of the container can be measured by removing measurement noise due to floating cells or floating colonies.

(Technique 4) The cell state measurement method according to Technique 3, wherein
the calculating the temporal variation characteristic evaluation value of the colony based on the first OCT signal within the determined calculation range includes:
calculating a temporal variation characteristic measurement value of the first OCT signal within the calculation range;
determining a position range of an individual colony based on the first OCT signal within the calculation range;
determining a scan position of the measurement light associated with the individual colony and a corresponding temporal variation characteristic measurement value based on the position range of the individual colony determined; and
acquiring correction coefficient data; and
applying a correction coefficient based on the acquired correction coefficient data to the temporal variation characteristic measurement value corresponding to the scan position associated with the individual colony to calculate the temporal variation characteristic evaluation value of the individual colony.

By this method, the cell state of a colony located near the wall surface of the container can be measured by calculating the temporal variation characteristic evaluation value of the colony.

(Technique 5) The cell state measurement method according to Technique 4, wherein
the acquiring correction coefficient data includes:
scanning the measurement light transmitted through the objective lens on the plane to illuminate an object including a plurality of partial zones arranged along the plane;
detecting a second OCT signal due to optical interference between a signal light that propagates along the optical axis due to reflection of the measurement light on the object and a reference light at a predetermined time interval;
calculating a second temporal variation characteristic measurement value of the second OCT signal corresponding to each of scanning positions of the measurement light based on the detected second OCT signal;
determining a position range of an individual partial zone in a planar coordinate system of the plane based on the second OCT signal;
determining a scanning position of the measurement light associated with the individual partial zone and a corresponding second temporal variation characteristic measurement value based on the determined position range of the individual partial zone; and
calculating a correction coefficient corresponding to a position coordinate of the first direction in the planar coordinate system based on the determined second temporal variation characteristic measurement value associated with the individual colony to form correction coefficient data.

By this method, the cell state of a colony located near the wall surface of the container can be measured by acquiring the correction coefficient data formed for an object and calculating the temporal variation characteristic evaluation value of the colony.

(Technique 6) The cell state measurement method according to Technique 5, wherein
the calculating the correction coefficient to form correction coefficient data includes:
determining a first position coordinate in the first direction of a first scan position and a first partial zone including the first scan position, in the planar coordinate system, based on the light intensity of the signal light forming the second OCT signal;
deeming the determined second temporal variation characteristic measurement value corresponding to the first scan position as a first temporal variation characteristic evaluation value of the first partial zone, calculating a first correction coefficient corresponding to the position coordinate in the first direction of each scan position associated with the first partial zone, so that the second temporal variation characteristic measurement value corresponding to each scan position associated with the first partial zone has a value equal to the first temporal variation characteristic evaluation value; and
forming correction coefficient data based on the calculated first correction coefficient, and recording the correction coefficient data.

By this method, the cell state of a colony located near the wall surface of the container can be measured by acquiring the correction coefficient data formed for an object and calculating the temporal variation characteristic evaluation value of the colony.

(Technique 7) The cell state measurement method according to Technique 6, wherein
the calculating the correction coefficient to form correction coefficient data further includes:
determining a second correction coefficient corresponding to a second position coordinate in the first direction of a second scanning position associated with a second partial zone that is different from the first partial zone based on the recorded correction coefficient data;
applying the determined second correction coefficient to the second temporal variation characteristic measurement value corresponding to the second scanning position to calculate a second temporal variation characteristic evaluation value of the second partial zone;
calculating a second correction coefficient corresponding to a position coordinate in the first direction of each scanning position associated with the second partial zone so that the second temporal variation characteristic measurement value corresponding to each scanning position associated with the second partial zone becomes a value equal to the second temporal variation characteristic evaluation value; and
adding the calculated second correction coefficient to the recorded correction coefficient data and recording the added correction coefficient data.

By this method, the cell state of a colony located near the wall surface of the container can be measured by acquiring the correction coefficient data formed for an object and calculating the temporal variation characteristic evaluation value of the colony.

(Technique 8) The cell state measurement method according to Technique 4, wherein
the acquiring the correction coefficient data includes:
acquiring the correction coefficient data recorded.

By this configuration, the cell state of a colony located near the wall surface of the container can be measured by acquiring the recorded correction coefficient data and calculating the temporal variation characteristic evaluation value of the colony.

(Technique 9) The cell state measurement method according to any one of Techniques 1 to 8, wherein
the calculating the temporal variation characteristic evaluation value further includes:
calculating a colony thickness in a direction orthogonal to the wall surface.

By this method, the temporal variation characteristic evaluation value of the colony and the colony thickness can be calculated, and the cell state of the colony located near the wall surface of the container can be three-dimensionally measured.

(Technique 10) A cell state measurement device for measuring a cell state of a colony in a container based on a temporal variation of signal intensity of an Optical Coherence Tomography (OCT) signal, including:
a light source device;
an optical system including an objective lens having an optical axis inclined with respect to a first direction along a plane parallel to a wall surface of the container on the plane, which is configured to divide a light emitted from the light source device into a measurement light and a reference light, scan the measurement light on the plane through the objective lens, irradiate a colony located near the wall surface with the measurement light through the wall surface, and irradiate the reference light on a reference surface;
a detector configured to detect an OCT signal due to optical interference between a signal light that propagates along the optical axis by reflection of the measurement light on the colony and a reference light reflected on the reference surface, at a predetermined time interval; and
an analyzer configured to calculate, based on the OCT signal detected by the detector, a temporal variation characteristic evaluation value of the colony corresponding to irradiation of the plane with a measurement light with an even light intensity.

By this configuration, the cell state of a colony located near the wall surface of the container can be measured.

(Technique 11) The cell state measurement device according to Technique 10, wherein the optical axis forms a first angle with respect to a direction orthogonal to the wall surface, and
the first angle is an angle equal to or greater than an opening angle of the objective lens and less than 90 degrees.

By this configuration, the influence of a specular reflection light from the wall surface can be suppressed, and the measurement accuracy of the colony near the wall surface can be improved.

Note that by appropriately combining discretionary exemplary embodiments among the various exemplary embodiments described above, effects of the exemplary embodiments can be achieved.

As described above, the accompanying drawings and the detailed description have been provided to describe the exemplary embodiments of the technique in the present disclosure. Thus, components described in the accompanying drawings and the detailed description may include not only components essential for solving the problem, but also components non-essential for solving the problem to describe the above techniques. For this reason, it should not be immediately recognized that these non-essential components are essential just because these non-essential components are described in the accompanying drawings and the detailed description.

Although the present disclosure has been fully described in connection with preferred exemplary embodiments with reference to the accompanying drawings, various modifications can be made within the scope of the claims. Such modifications and exemplary embodiments obtained by appropriately combining technical units disclosed in different exemplary embodiments are also included in the technical scope of the present disclosure.

According to an embodiment of the present disclosure, the cell state of a colony located near the wall surface of the container can be measured.

### INDUSTRIAL APPLICABILITY

The present disclosure is applicable to the measurement of the cell state of a colony during cell culture. The present disclosure is useful for measuring, monitoring, and the like a state of a cell cultured body in a cell culture device.

What is claimed is:

1. A cell state measurement method for measuring a cell state of a colony in a container by a temporal variation of a signal intensity of an Optical Coherence Tomography (OCT) signal, the cell state measurement method comprising:
scanning a measurement light transmitted through an objective lens having an optical axis inclined with respect to a first direction along a plane parallel to a wall surface of the container on the plane, and irradiating a colony, which is located near the wall surface, with the measurement light through the wall surface;
detecting a first OCT signal due to optical interference between a signal light that propagates along the optical axis by reflection of the measurement light on the colony and a reference light, at a predetermined time interval; and
calculating, based on the detected first OCT signal, a temporal variation characteristic evaluation value of the colony corresponding to irradiation of the plane with a measurement light having an even light intensity.

2. The cell state measurement method according to claim 1,
wherein the optical axis forms a first angle with respect to a direction orthogonal to the wall surface, and
the first angle is an angle equal to or greater than an opening angle of the objective lens and less than 90 degrees.

3. The cell state measurement method according to claim 2,
wherein the calculating the temporal variation characteristic evaluation value comprises:
determining a calculation range from the wall surface toward the inside of the container in a direction orthogonal to the wall surface; and
calculating the temporal variation characteristic evaluation value of the colony based on the first OCT signal within the calculation range determined in determining.

4. The cell state measurement method according to claim 3, wherein
the calculating the temporal variation characteristic evaluation value of the colony based on the first OCT signal within the determined calculation range comprises:
calculating a temporal variation characteristic measurement value of the first OCT signal within the calculation range;
determining a position range of an individual colony based on the first OCT signal within the calculation range;
determining a scan position of the measurement light associated with the individual colony and a corresponding temporal variation characteristic measurement value based on the position range of the individual colony determined; and
acquiring correction coefficient data; and
applying a correction coefficient based on the acquired correction coefficient data to the temporal variation characteristic measurement value corresponding to the scan position associated with the individual colony to calculate the temporal variation characteristic evaluation value of the individual colony.

5. The cell state measurement method according to claim 4, wherein
the acquiring correction coefficient data comprises:
scanning the measurement light transmitted through the objective lens on the plane to illuminate an object including a plurality of partial zones arranged along the plane;
detecting a second OCT signal due to optical interference between a signal light that propagates along the optical axis due to reflection of the measurement light on the object and a reference light at a predetermined time interval;
calculating a second temporal variation characteristic measurement value of the second OCT signal corresponding to each of scan positions of the measurement light based on the detected second OCT signal;
determining a position range of an individual partial zone in a planar coordinate system of the plane based on the second OCT signal;
determining a scan position of the measurement light associated with the individual partial zone and a corresponding second temporal variation characteristic measurement value based on the determined position range of the individual partial zone; and
calculating a correction coefficient corresponding to a position coordinate of the first direction in the planar coordinate system based on the determined second temporal variation characteristic measurement value associated with the individual colony to form correction coefficient data.

6. The cell state measurement method according to claim 5, wherein
the calculating the correction coefficient to form correction coefficient data comprises:
determining a first position coordinate in the first direction of a first scan position and a first partial zone including the first scan position, in the planar coordinate system, based on the light intensity of the signal light forming the second OCT signal;
deeming the determined second temporal variation characteristic measurement value corresponding to the first scan position as a first temporal variation characteristic evaluation value of the first partial zone, calculating a first correction coefficient corresponding to the position coordinate in the first direction of each scan position associated with the first partial zone, so that the second temporal variation characteristic measurement value corresponding to each scan position associated with the first partial zone has a value equal to the first temporal variation characteristic evaluation value; and
forming correction coefficient data based on the calculated first correction coefficient, and recording the correction coefficient data.

7. The cell state measurement method according to claim 6, wherein
the calculating the correction coefficient to form correction coefficient data further comprises:
determining a second correction coefficient corresponding to a second position coordinate in the first direction of a second scan position associated with a second partial zone that is different from the first partial zone based on the recorded correction coefficient data;
applying the determined second correction coefficient to the second temporal variation characteristic measurement value corresponding to the second scan position to calculate a second temporal variation characteristic evaluation value of the second partial zone;
calculating a second correction coefficient corresponding to a position coordinate in the first direction of each scan position associated with the second partial zone so that the second temporal variation characteristic measurement value corresponding to each scan position associated with the second partial zone becomes a value equal to the second temporal variation characteristic evaluation value; and
adding the calculated second correction coefficient to the recorded correction coefficient data and recording the added correction coefficient data.

8. The cell state measurement method according to claim 4, wherein
the acquiring the correction coefficient data comprises:
acquiring the correction coefficient data recorded.

9. The cell state measurement method according to claim 1, wherein
the calculating the temporal variation characteristic evaluation value further comprises:
calculating a colony thickness in a direction orthogonal to the wall surface.

10. A cell state measurement device for measuring a cell state of a colony in a container based on a temporal variation of signal intensity of an Optical Coherence Tomography (OCT) signal, comprising:
a light source device;
an optical system comprising an objective lens having an optical axis inclined with respect to a first direction along a plane parallel to a wall surface of the container on the plane, which is configured to divide a light emitted from the light source device into a measurement light and a reference light, scan the measurement light on the plane through the objective lens, irradiate a colony, which is located near the wall surface, with the measurement light through the wall surface, and irradiate the reference light on a reference surface;
a detector configured to detect an OCT signal due to optical interference between a signal light that propagates along the optical axis by reflection of the measurement light on the colony and a reference light reflected on the reference surface, at a predetermined time interval; and
an analyzer configured to calculate, based on the OCT signal detected by the detector, a temporal variation characteristic evaluation value of the colony corresponding to irradiation of the plane with a measurement light with an even light intensity.

11. The cell state measurement device according to claim 10,
wherein the optical axis forms a first angle with respect to a direction orthogonal to the wall surface, and
the first angle is an angle equal to or greater than an opening angle of the objective lens and less than 90 degrees.
